(19) United States
(12) Patent Application Publication
(10) Pub. No.: US 2016/0272934 A1
(43) Pub. Date: Sep. 22, 2016

(54) SYSTEMS, DEVICES AND METHODS FOR MICROFLUIDIC CULTURING, MANIPULATION AND ANALYSIS OF TISSUES AND CELLS

(71) Applicant: CELLANYX DIAGNOSTICS, LLC, Beverly, MA (US)

(72) Inventors: Ashok C. CHANDER, Boston, MA (US); Wendell R. SU, Taipei City (TW); Thiagarajan MEYYAPPAN, Foxboro, MA (US); Jonathan VARSANIK, Brookline, MA (US); Senthil RAMU, Boston, MA (US); Michael MANAK, Milton, MA (US); Brad HOGAN, Somerville, MA (US)

(73) Assignee: CELLANYX DIAGNOSTICS, LLC, Beverly, MA (US)

(21) Appl. No.: 15/031,074

(22) PCT Filed: Oct. 22, 2014
(86) PCT No.: PCT/US2014/061782

§ 371 Date: Apr. 21, 2016

Related U.S. Application Data
(60) Provisional application No. 61/894,298, filed on Oct. 22, 2013.

(51) Int. Cl. C12M 1/00; G01N 1/40

(52) U.S. Cl. CPC C12M 47/04; G01N 1/4077; C12M 23/22; C12M 29/10

(57) ABSTRACT

Microfluidic devices for dissociating tissue, culturing, separating, manipulating, and assaying cells and methods for manufacturing and using the devices are disclosed. Individual modules for tissue dissociation, cell, protein and particle separation, cell adhesion to functionalized, permissive micro- and nano-substrates, cell culturing, cell manipulation, cell and extracellular component assaying via metabolic and therapeutic compounds are described. Specialized micro- and nano-substrates and their methods of fabrication are also described. An integrated device is also disclosed. The devices and methods can be used for diagnostic applications, monitoring of disease progression, analysis of disease recurrence, compound discovery, compound validation, drug efficacy screening, and cell-based assays.

### CROSS-REFERENCE TO RELATED APPLICATIONS

This application claims priority to U.S. Provisional App. No. 61/894,298, filed Oct. 22, 2013. This application is related to U.S. application Ser. No. 13/682,710, filed Nov. 20, 2012, which claims priority to U.S. Provisional App. No. 61/561,907, filed Nov. 20, 2011, and U.S. Provisional App. No. 61/677,157, filed Jul. 30, 2012. This application is also related to International App. No. PCT/US2011/055444, filed Oct. 7, 2011 and designating the U.S., which claims priority to U.S. Provisional App. No. 61/391,340, filed Oct. 8, 2010. The teachings of each of these applications are incorporated by reference.

### FIELD

Systems, methods, and devices related to the field of medical testing/diagnostics, cell-based assays, and compound discovery are provided herein. In various aspects, systems, devices, and methods are provided for the determination of the growth, and/or oncogenic potential, migration rate, and/or metastatic potential of mammalian cells or patient's cells (e.g., cells obtained from biopsy). In some aspects, microfluidic tissue disassociation, cell, protein, and particle separation, cell manipulation, and assay devices and methods for using the same are provided. Exemplary applications include but are not limited to diagnostic and cell based assays.

### BACKGROUND

Primary cell culture that allows the study of native tissue samples derived from an organism. Culturing cells derived from organisms, can be useful and necessary for applications such as medical diagnostics, cell-based assays, for compound discovery and characterization.

For example, cancer diagnosis and identification of compounds for treatment of cancer are of great interest due to the widespread occurrence of the diseases, high death rate, and recurrence after treatment. According to National Vital Statistics Reports, from 2002 to 2006 the rate of incidence (per 100,000 persons) of cancer in Caucasians was 470.6, in people of African descent 493.6, in Asians 311.1, and Hispanics 350.6, indicating that cancer is wide-spread among all races. Lung cancer, breast cancer and prostate cancer were the three leading causes of death in the US, claiming over 227,900 lives in 2007 according to the NCI.

Survival of a cancer patient depends heavily on detection. As such, developing technologies applicable for sensitive and specific methods to detect cancer is an inevitable task for cancer researchers. Existing cancer screening methods include: 1. the Papanicolau test for women to detect cervical cancer and mammography to detect breast cancer, 2. prostate-specific antigen (PSA) level detection in blood sample for men to detect prostate cancer, 3. occult blood detection for colon cancer, and 4 endoscopy, CT scans, X-ray, ultrasound imaging and MRI for various cancer detection. These traditional diagnostic methods however are not very powerful when it comes to cancer detection at very early stages and give little prognostic information. Moreover, some of the screening methods are quite costly and not available for many people.

Likewise, existing methods for cancer staging are often qualitative and therefore limited in applicability. For example, diagnoses made by different physicians or of different patients using existing methods can be difficult to compare in a meaningful manner due to the subjective nature of these methods. As a result, the subjectivity of the existing methods of cancer staging often results in overly aggressive treatment strategies. By way of example, in the absence of better data, the most drastic, potentially invasive, strategy is often recommended, which can lead to overtreatment, poor patient quality of life, and increased medical costs.

One method to detect and/or characterize cancer, for example, is to directly assess living tissue derived from small biopsy samples taken from suspicious tissue. To get a relevant and useful sense of the biological characteristics of tissue, one would be well served by being able to culture biopsy tissue in vitro.

Therefore, the development of technology that is specific and reliable for culturing primary human tissue and/or detecting and characterizing a cancer (e.g., determining the growth, oncogenic, migration rate, and/or metastatic potential of cells obtained from a patient) is an area of significant importance. Likewise, there remains a need for improved systems, methods, and devices for diagnostic cell-based assays and compound discovery.

### SUMMARY

The systems, methods, and devices described herein generally involve medical testing/diagnostics, cell-based assays, and/or compound discovery. In various aspects, microfluidic devices, systems, and methods disclosed herein can provide clinical and/or research purposed diagnostics and assay platforms that enable tissue disassociation, cell, protein, and particle separation, and cell manipulation. The systems and devices disclosed herein can provide, for example, the culturing of a small number of cells in environments that can approximate in vivo conditions, while allowing for a determination of the cells' growth, and/or oncogenic potential, migration rate, and/or metastatic potential. A determination of these characteristics can, among other things, facilitate treatment decision steps taken by a physician for patients having symptoms of cancer and/or aid in the discovery of therapeutics that alter and/or perturb a cell's characteristics that engender its cancer-like, oncogenic, and/or metastatic phenotype.

For example, quantitative prognostic metrics according to aspects of the present disclosure can improve the accuracy of diagnosis by supplementing a physician's decision-making process with clinical data to support the available treatment options. As a result, embodiments of the present disclosure can provide numerous advantages, for example, reduced healthcare costs, reduced risk associated with treatment, improved patient quality of life, and increased patient survival.

As will be described in detail below, one exemplary aspect of the present disclosure provides cell processing systems and devices, including uses thereof, that include microfluidic channels and a substrate to process (e.g., culture, filter, image) cells derived, for example, from a biopsy. In other aspects, the systems and devices enable diagnostic imaging, cell-based assays such as metabolic testing, and/or compound discovery.

In one exemplary embodiment, a system for cell processing is provided. The system can include at least one microfluidic cell dissociation module and at least one microfluidic cell-processing module fluidly coupled to at least one cell dissociation module. The cell dissociation module can be configured, for example, to dissociate one or more tissue fragments received therein into one or more of single cells and/or smaller tissue fragments. The microfluidic cell-processing module can receive at least a portion of said one or more single cells and/or smaller tissue fragments.

In various embodiments, one or more cell-processing modules of the system can be configured to perform various cell processing functions. In various aspects, microfluidic systems can incorporate one or more of the following exemplary individual microfluidic modules and/or substrates:
a cell dissociation module, which can receive mammalian tissue and separate the tissue into smaller clumps and/or single cells, e.g., via enzymatic, mechanical, and/or shear forces;
a cell separation module, where cells and extra-cellular components such as proteins and other particles can be segregated and sorted;
perfusion chambers, in which single cells and/or clumps of cells can be adhered to specialized micro- and nano-featured substrates. When functionalized with protein coatings, these specialized substrates can create a permissive surface for cell adhesion and subsequent examination via microscopy techniques. Cells can also be cultured in such an environment;
metabolic assay, compound discovery, and titration modules integrated as part of the above perfusion chamber, whereby cells adhered to various substrates can be subjected to various compounds for assay or therapeutic applications. The cells can then be monitored via microscopy techniques for their response. Titrations can also be conducted in the titration module and can be similarly inspected via microscopy; and
various specialized substrates for cell adhesion and also for testing cellular properties such as invasion potential.

In frequent embodiments, an extracellular matrix (ECM) formulation is used to functionalize the microfluidic device or substrates contained therein. The formulation, for example, improves primary cell culture growth conditions by closely replicating an in vivo environment (rather than a plain polymer substrate to which cells often poorly attach). This allows cells from biopsy samples to be cultured in vitro. In their natural in vivo environments, cells interact with other cells and the surrounding ECM. A cell's external environment can greatly influence it. Additionally, the mechanisms by which cells respond to external stimuli shed light onto the properties of a cell's underlying state. For example, cells can sense the stiffness of their surroundings and induce distinct and irreversible remodeling of the ECM and ECM-cell contacts. Overall tissue stiffness or tissue stiffness gradients can be integral in tumor progression and other diseases.

In an exemplary embodiment, a cell dissociation module can include one inlet port for receiving one or more tissue fragments, several cell dissociation chambers, an outlet port for extracting fluids, dissociated cells, and other particles to be transmitted to one or more downstream modules for further processing, and a channel fluidly coupled to said chambers, inlet, and an outlet to allow fluid to be displaced through the chamber. A pump can be coupled to the inlet or outlet to cause movement of the fluid through the channel and/or chambers.

The cell dissociation module can have various configurations and dimensions. By way of example, the fluidic pathway can take on a serpentine, spiral shape, or configured for back and forth movement of the fluid within a dissociation chamber. In some aspects, a plurality of microstructures disposed in the channel can facilitate dissociation of said one or more tissue fragments. The microstructures can have a variety of dimensions. By way of example, the microstructures can range from about 1 micron to 1 millimeter in length and vary in post-to-post gap distance from 100 micron to 1 mm. The microstructures can have a variety of configurations to facilitate dissociation, e.g., through mechanical perturbation. For example, the microstructures can be rectangular.

As noted above, systems and devices in accord with the present teachings can include a cell-processing module. In one exemplary embodiment, the cell-processing module includes a perfusion chamber to culture cells, image samples, and perform metabolic assays. The chamber can include a sample inlet for receiving one or more samples, a reagent inlet for receiving one or more metabolic reagents, an imaging/culturing chamber for culturing, imaging, and analyzing cells, and a waste outlet for extracting waste fluid, and a channel fluidly coupled to said chamber, inlets, and outlet to allow fluid to be displaced through the chamber.

In some embodiments, the cell-processing module can be adapted for hands free, long term cell culture by coupling a media reservoir to the imaging/culture chamber. In addition, the channels entering the imaging/culturing chamber can have baffles to encourage even flow distribution within said channel.

In various aspects, the perfusion chamber can take on various configurations and dimensions. By way of examples, the imaging/culturing chamber can be hexagonal, and the module can have a number of perfusion chambers for parallel processing.

In another exemplary embodiment, the cell-processing module can perform adhesion-based cell sorting of heterogeneous cell populations. By way of example, cell sorting is performed by fluidly linking a number of perfusion chambers from the above description, each perfusion chamber functionalized with a different type of substrate with micro-and nano-features coated with proteins solutions specialized for attachment of one particular subset of cells in the heterogeneous mixture of cells in the sample. In another example, the cell sorting module consists of several sorting chambers, a cell inlet, several reagent inlets, and several waste outlets. The chambers are also functionalized with different types of substrates. In some aspects, the cell-sorting module can be adapted for cell culturing, imaging, and metabolic assays.

In some aspects, the various modules described herein (or at least a portion of the modules such as the microfluidic channels) can be formed in a monolithic substrate. For example, a cell-dissociation module and the cell-processing layer of a cell-processing module can be formed in a monolithic substrate. Such devices can be fabricated and operated with techniques familiar to those skilled in the art of multi-layer soft lithography, photolithography, and microfluidic device fabrication and in light of the teachings herein.

In addition or in the alternative, because discrete functions can be performed by the one or more modules, individual modules can be coupled to one another and/or combined to create an integrated chip or platform that can be used for numerous biological and chemical applications, for example, but not limited to a cell-based assay for compound discovery, validation, testing, and or an in vitro diagnostic or prognostic test for disease states such as epithelial-born cancers, blood-born cancers, bone cancer, skin cancer, lung cancer, prostate cancer, breast cancer, pancreatic cancer, brain cancer, cervical cancer, colon cancer, stomach cancer, cardiac hypertrophy, cardiovascular diseases, and fibrotic diseases such as fibrosis of the kidney, and liver.

By disassociating and or culturing tissue and cells derived from an organism using any combination of the devices and substrates described herein, it can be possible to create powerful experimental and diagnostic tools with immediate research, pharmaceutical, biotechnology, and clinical development applications.

In accordance with various aspects of the present teachings, methods for producing the exemplary devices and systems described herein are also provided. By way of example, in some aspects a method of manufacturing is provided by producing a rigid substrate within at least one of the imaging and sorting chambers having a fixed height. In a related aspect, the rigid substrate can comprise a plurality of microstructures. In various aspects, the rigid substrate can be produced using photo-polymerization. In accord with various aspects, rigid substrate having regions of different stiffness can be produced within an imaging chamber, for example, by modulating the intensity of light during the photo-polymerization process. Additionally or alternatively, a rigid substrate can be produced in a two layer imaging chamber, for example, through the use of a dissolvable membrane.

These and other embodiments, features, and advantages will become apparent to those skilled in the art when taken with reference to the following more detailed description of various exemplary embodiments of the present disclosure in conjunction with the accompanying drawings.

### BRIEF DESCRIPTION OF THE DRAWINGS

The skilled person in the art will understand that the drawings, described below, are for illustration purposes only.

FIG. 1 is a schematic representation of a microfluidic tissue dissociation chamber;

FIG. 1A is a schematic representation of a microfluidic tissue dissociation chamber;

FIG. 2 is a schematic representation of a microfluidic tissue dissociation chamber;

FIG. 3 schematic representation of a microfluidic device designed for the introduction and adhesion of cells for imaging of cells via techniques such as optical-based microscopy, and introduction of reagents to perform various biochemical assays;

FIG. 3A schematic representation of a microfluidic device designed for the introduction and adhesion of cells for imaging of cells via techniques such as optical-based microscopy, and introduction of reagents to perform various biochemical assays;

FIG. 3B schematic representation of a microfluidic device designed for the introduction and adhesion of cells for imaging of cells via techniques such as optical-based microscopy, and introduction of reagents to perform various biochemical assays;

FIG. 4 is a schematic representation of a microfluidic device designed for the introduction and adhesion of cells for imaging of cells via techniques such as optical-based microscopy, introduction of reagents to perform various biochemical assays, and long term culturing of cells;

FIG. 4A is a schematic representation of a microfluidic device designed for the introduction and adhesion of cells for imaging of cells via techniques such as optical-based microscopy, introduction of reagents to perform various biochemical assays, and long term culturing of cells;

FIG. 5 is a schematic representation of a microfluidic device enabling adhesion-based cell sorting from a heterogeneous population of cells, imaging of cells via techniques such as optical-based microscopy, and introduction of reagents to perform various biochemical assays;

FIG. 6 is a schematic representation of a microfluidic device enabling adhesion-based cell sorting from a heterogeneous population of cells;

FIG. 7 is a schematic representation of an integrated microfluidic device featuring inlets, tissue dissociation module, cell sorting module, and perfusion array;

FIG. 8 is a schematic representation of an integrated microfluidic device featuring inlets, tissue dissociation module, cell sorting module, and perfusion array;

FIG. 9 is a schematic representation of a multilayered, integrated microfluidic device featuring inlets, tissue dissociation module, cell sorting module, flow dividing module, cell imaging module, and outlets;

FIG. 9A is a schematic representation of a microfluidic tissue dissociation module

FIG. 9B is a schematic representation of a microfluidic device enabling adhesion-based cell sorting from a heterogeneous population of cells;

FIG. 9C is a schematic representation of a microfluidic device enabling division of a single sample source into multiple samples of smaller volumes;

FIG. 9D is a schematic representation of a microfluidic device enabling imaging of cells;

FIG. 9E is a schematic representation of a microfluidic device enabling collection of waste fluid;

FIG. 10 is a schematic representation of reversible connections of one microfluidic device to another in the z-direction;

FIG. 11 is a schematic representation of a multilayered, integrated microfluidic device featuring inlets, tissue dissociation module, cell sorting module, flow dividing module, cell imaging module, and outlets;

FIG. 11A is a schematic representation of an integrated microfluidic device enabling tissue dissociation and cell sorting; and

FIG. 11B is a schematic representation of an integrated microfluidic device enabling sample dividing and cell imaging;

FIG. 12 is an exemplary process for producing rigid substrates in accordance with various aspects of the applicant's present teachings; and

FIGS. 13 and 13A depict a side view and top view, respectively, of an exemplary microfluidic chip produced utilizing an exemplary method for substrate polymerization in accordance with various aspects of the present teachings.

FIG. 14 presents an exemplary curve of adherent cell numbers versus time, indicating that devices of the present disclosure can process diagnostic results within 3 days of receiving sample.

FIG. 15 depicts a variety of the aspects capable of interrogation using the devices and according to the methodologies described herein, which presents an innovative suite of biomarkers to better assess tumor aggressiveness and biological behavior leading to improved patient risk stratification.

FIG. 16 presents exemplary data indicating that oncologic potential and metastatic potential are used to distinguish between normal and malignant tissue and low- and high-risk patient samples by analyzing individual cells within a sample. (A) represents cell distribution for a normal tissue, and (B) represents cell distribution for a malignant tissue.

FIG. 17 presents a chart indicating the stratification of patients into 4 zones, predicting indolent (PxP Zone 1), local growth potential (PxP Zone 2), metastatic potential (PxP Zone 3), and both local growth and metastatic growth potential (PxP Zone 4).

FIG. 18A depicts (A) the distribution of a number of samples on the OP3 vs MP2 algorithm graph, and (B) is a closeup of the boundaries of the indolent/malignant zones.

FIG. 18B depicts (A) the plot of a number of samples determined to be negative for seminal vesicle invasion, and (B) a number of samples determined to be positive for seminal vesicle invasion.

FIG. 18C depicts (A) the plot of a number of samples determined to not have positive margins during surgery, and (B) a number of samples with positive margins during surgery.

FIG. 18D depicts (A) the plot of a number of samples that do not exhibit vascular invasion, and (B) a number of samples which exhibit vascular invasion.

### DETAILED DESCRIPTION OF THE VARIOUS EMBODIMENTS

As used herein, the term “a” or “an” means “at least one” or “one or more.”

As used herein, “Oncogenic potential” (OP) refers to a quantitative prediction of a tumor's growth potential.

As used herein “Metastatic potential” (MP) refers to a quantitative prediction of whether a tumor will invade other tissues.

Any sample suspected of containing cells relevant to the therapeutic indication being evaluated can be utilized in the devices and according to the methods of the present disclosure. By way of non-limiting example, the sample may be tissue (e.g., a prostate biopsy sample or a tissue sample obtained by prostatectomy), blood, urine, semen, cells (such as circulating tumour cells), cell secretions or a fraction thereof (e.g., plasma, serum, exosomes, urine supernatant, or urine cell pellet). In the case of a urine sample, such is often collected immediately following an attentive digital rectal examination (DRE), which causes prostate cells from the prostate gland to shed into the urinary tract. The patient sample may require preliminary processing designed to isolate or enrich the sample for the markers or cells that contain the markers. A variety of techniques known to those of ordinary skill in the art may be used for this purpose. As used herein “sample” is used generically and is intended to refer to any of a raw patient sample, a preliminarily processed sample, and/or a processed patent sample, including that derived from prostate, bladder, colon, breast, lung, kidney, or another tissue.

The following detailed description should be read with reference to the drawings. The drawings, which are not necessarily to scale, depict selected embodiments and are not intended to limit the scope of the present disclosure. The detailed description illustrates by way of example, and is not intended to limit the scope of the present disclosure.

The teachings herein generally provide microfluidic systems, devices, and methods for dissociating, culturing, assaying, inspecting, and/or otherwise manipulating cells, and can have application in medical testing/diagnostics, cell-based assays, and/or compound discovery. In various aspects, the exemplary microfluidic devices, systems, and methods can provide clinical and research purposed diagnostics and assay platforms that enable tissue disassociation, cell, protein, and particle separation, and other cell manipulation. By way of example, the present teachings can enable the culturing of a small number of cells in environments that can approximate in vivo conditions, while allowing for a determination of the cells' growth, and/or oncogenic potential, migration rate, and/or metastatic potential.

As will be described in detail below, exemplary cell processing systems and methods in accordance with the present teachings enable a variety of cell processing procedures, cell-based assays, and/or experiments (e.g., compound discovery) to be performed within the various microfluidic modules described in detail below. Though particular cell-processing functions are generally described with reference to individual cell-processing modules, it will be appreciated that the various exemplary modules and/or their functions can be combined to form a cell-processing system for performing multiple cell-processing functions. By way of example, it will be appreciated that various exemplary modules described herein can be combined in a single device (e.g., in a lock-and-key manner or combined in a monolithic microfluidic chip) to enable a specific clinical, diagnostic, and/or experimental workflow. Accordingly, the following description provides exemplary modules that can be incorporated into various systems in accord with the present disclosure.

### Tissue Dissociation

In one aspect, a microfluidic tissue dissociation module can be provided. Tissue dissociation involves in the progressive isolation of smaller and smaller clusters of tissue and cell clumps into a single cell suspension. The process of dissociation can be accomplished via anumber of methods and combinations of methods including, but not limited to, enzymatic treatment, mechanical agitation, stress, and shear forces.

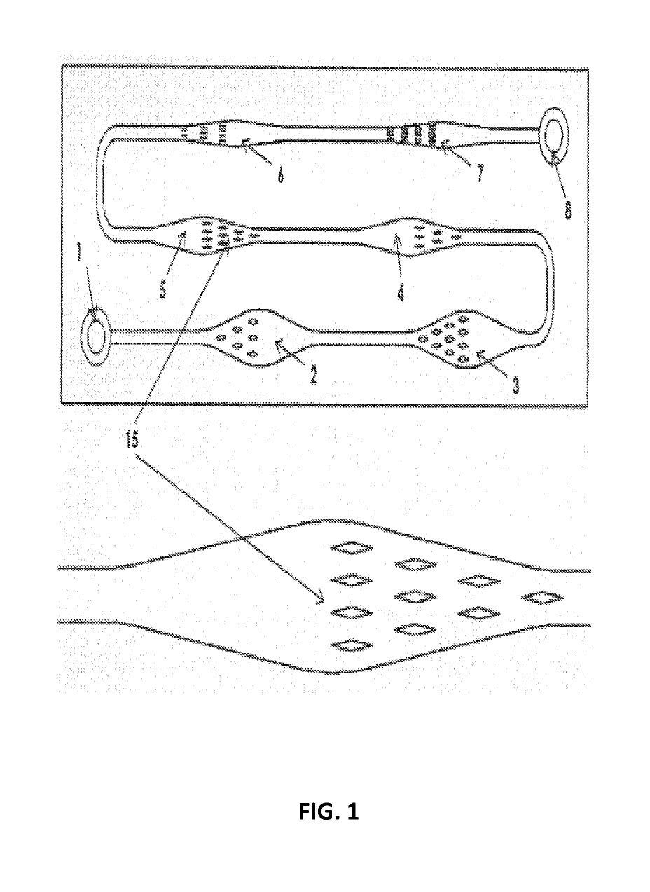

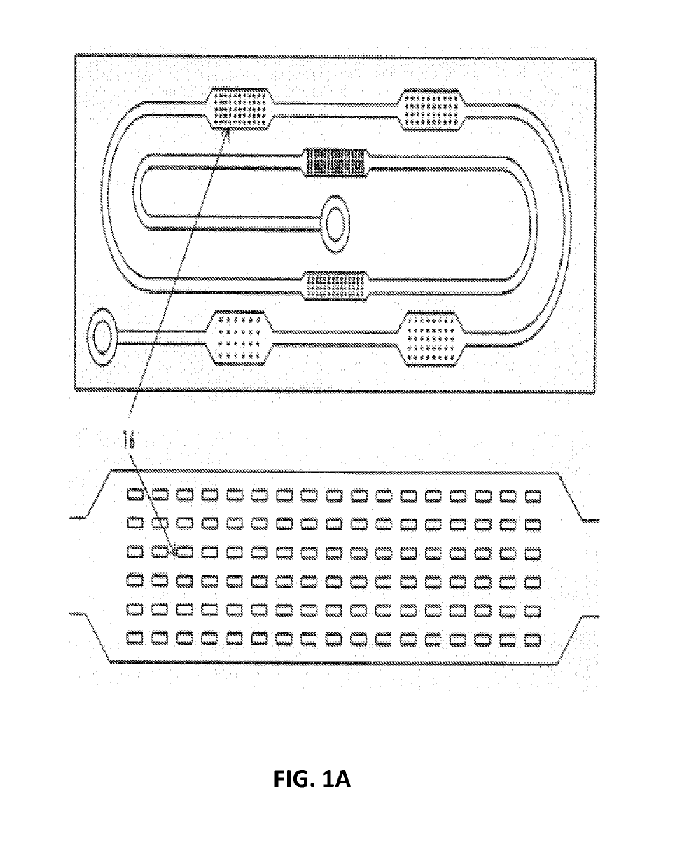

Schematic representations of microfluidic devices for tissue dissociation are shown in FIG. 1, 1A, and 2. In an exemplary depiction, as seen in FIG. 1, tissue fragments (e.g., minced tissue, sliced biopsy tissue, etc) can be inserted in inlet port 1. The tissue fragments may be mixed beforehand with dissociation enzymes such as trypsin, DNase, papain, collagenase type I, II, III, IV, hyoluronidase, elastase, protease type XIV, pronase, dispase I, dispase II, and neutral protease. As will be appreciated by a person skilled in the art, the tissue fragments can range in size, for example, in a range of about 0.1 mm to 1 mm. In some aspects, tissue samples can be injected into the device via needles, pipettes, or integrated and exterior fluidic handling mechanisms, such as plastic tubes. Six dissociation chambers 2 through 7 are utilized to reduce the input tissue samples to progressively smaller clumps of tissue until ultimately an approximately single cell suspension is achieved. As will be appreciated by a person skilled in the art, 6 dissociation chambers are shown here, although more or less chambers (e.g., n) are possible. The dissociated cells can then be extracted from outlet 8 via the same, similar, or different methods from those used to introduce the samples.

Once loaded within the device, a positive pressure from inlet 1 or a vacuum source from outlet 8 can be used to displace the fluid mixture within the device at varying flow rate. As will be appreciated by a person skilled in the art, any pressure generators known or developed hereafter and modified in accord with the teaching herein can be utilized to displace the fluid mixture within the device.

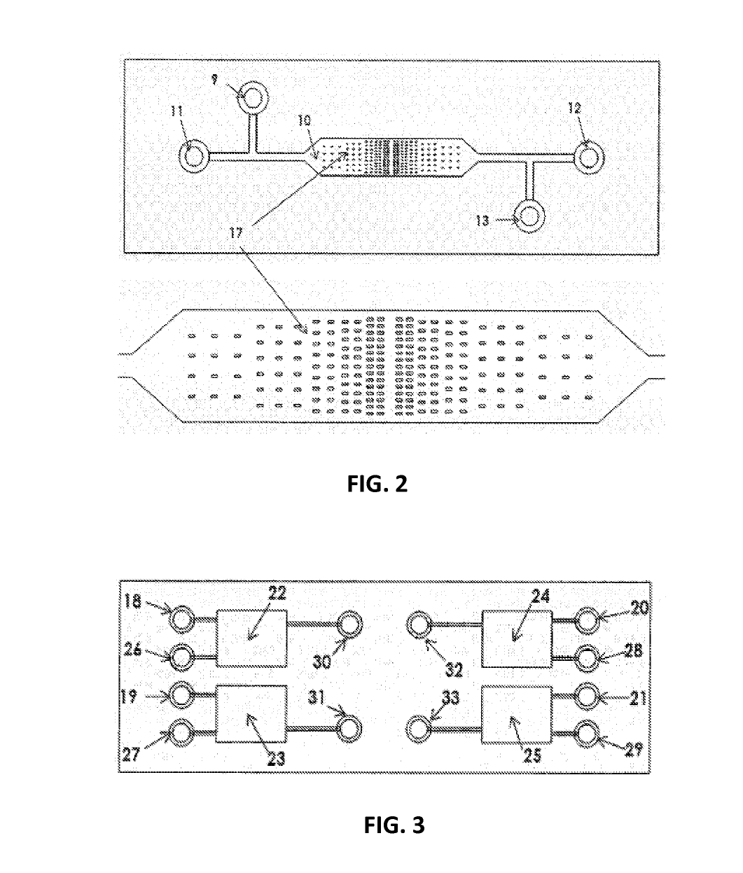

As will be appreciated by a person skilled in the art, the fluidic pathway of the tissue dissociation module may take on different configurations to transport the tissue samples and associated fluid to the various dissociation chambers depending on the usable space available on the microfluidic chip. By way of example, the fluidic pathway of 1A takes on a spiral shape as opposed to the serpentine shape as depicted in FIG. 1. In another exemplary depiction, as seen in FIG. 2, the tissue fragments can enter the device via any port, including sample inlet 9, and pass back and forth through dissociation chamber 10 via alternating positive pressure source between pressure inlets 11 and 12. The dissociated cells can then be extracted from any port, including cell outlet 13.

With reference now to FIGS. 1, 1A, and 2, various microstructures can be incorporated into the dissociation module to facilitate mechanical perturbation. In the depicted embodiments, for example, a plurality of microstructures is present at the top or bottom of the chamber to aid in the mechanical perturbation of the tissue samples. As will be appreciated by a person skilled in the art, microstructures 15, 16, and 17 can have various dimensions, configurations, and geometries. By way of example, the microstructure 17 can range from about 1 micron to 1 millimeter in length and vary in post-to-post gap distance from 100 micron to 1 mm. Their presence, along with the varying chamber geometries, flow rates, and finally the presence of dissociation enzymes in the formulation, can enable the reduction of tissue fragments to single cells.

Upon completion of tissue dissociation into single cells or small clumps of cells, the dissociation module can, for example, transfer the cells and the associated fluid and other particles to one or more downstream modules for further processing such as cell sorting, culturing, adhesion, and imaging via one or more of the various embodiments described below.

Perfusion Chamber for Metabolic assays, Cell Culturing, and Imaging.

As noted above, various cell-processing modules can be utilized to perform various functions. In various exemplary embodiments, a perfusion chamber module can be provided. Specialized substrates with micro-and nano-features coated with proteins solutions such as fibronectin, laminin, vitronectin, and collagen can be incorporated within the perfusion chamber module so as to create permissive surfaces upon which mammalian cells can preferentially adhere and be cultured when they otherwise would be unable. Examples of these substrates are described, for example in PCT/US2011/055444 filed Oct. 7, 2011, the contents of which are incorporated herein by reference.

Several of biomarkers noted above are based on cell-ECM interactions. In those interactions, the primary mechanotransducers between the cell and the ECM are integrins. The integrins recruit proteins to the sites of cell-ECM contacts, forming aggregates known as focal adhesions. The adhesions connect the external matrix to the cytoskeletal structure of the cell. Both focal adhesion proteins and integrins have been shown to be involved in the ontogeny of many epithelial cancers, including prostate cancer or cancer of or derived from colon, breast, lung, kidney, or bladder tissues. Focal adhesion proteins and integrins also participate in cell-ECM mediated events, and ECM additionally plays a role in the development of diseases. In addition, preclinical data and other emerging evidence suggest that focal adhesion-actin coupling regulates more than simple motility events. The coupling may, for example, regulate cellular growth and proliferation. In examining cell-ECM interactions overall, frequent biomarkers are related to or provide measures of focal adhesion, actin dynamics, and/or cellular force generation. Research indicates that metrics derived from these biomarkers are able to differentiate between healthy and cancer cells. One such metric comprises a Traction Force Index (TFI). This metric was measured in multiple cell lines: including healthy, wild-type human cell lines and human cancer cell lines. When plotted against doubling time and migration rate, TFI has approximately linear and parabolic correlations, respectively. The inventors have found that healthy cell types exhibit low values (<10), while cancer cell lines exhibited higher ones (>10).

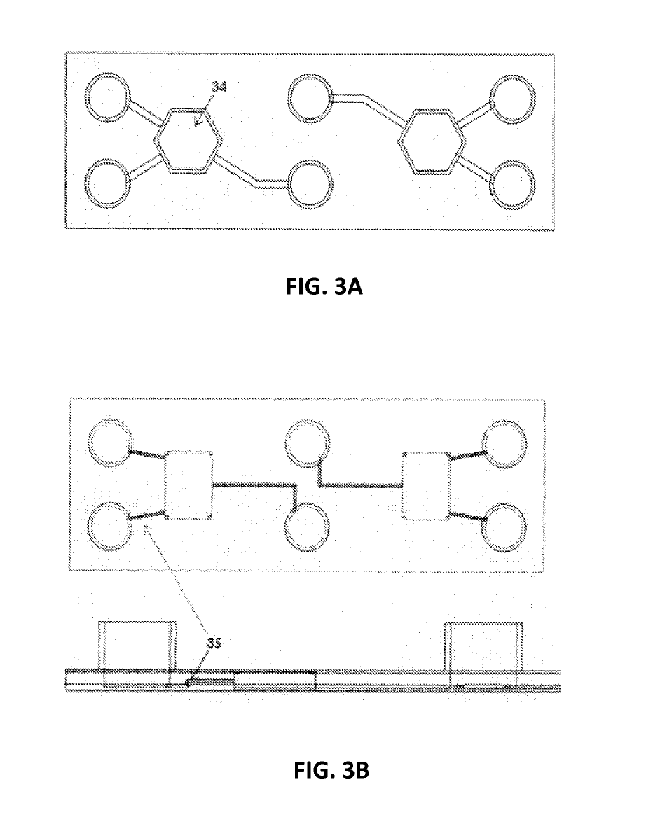

With reference now to the exemplary module depicted in FIG. 3, the single perfusion chamber can enable the introduction of mammalian cells through sample inlets 18, 19, 20, and 21, their adhesion to the specialized substrate region in the imaging/culturing chamber 22, 23, 24, and 25, introduction of various reagents for metabolic assays through reagent inlets 26, 27, 28, and 29, and subsequent inspection via techniques such as optical-based microscopy. Waste fluid can be collected and removed via waste outlets 30, 31, 32, and 33. As noted above, this substrate region can be comprised of micro- and nano-structures to enable investigation of the cell's characteristics (e.g. motility). Examples of fabrication methods, materials, and dimensions are described in PCT/US2012/066162 filed Nov. 20, 2012, the contents of which are incorporated herein by reference.

On an exemplary device, cells are seeded, grown, and imaged with limited operator interaction. Prior to seeding cells, the device is often sterilized and coated with an ECM formulation. Selected biomarkers are periodically measured, for example, via microscopy. In certain embodiments, a single device can simultaneously analyze 1000 cells. The device's structure also often plays a role in biomarker measurement. The structure is preferably comprised of substrates, and the stiffness of a substrate is often controlled, for example, through the use of micropillars. One biomarker, cellular force generation, can be measured by analyzing the deflection of a micropillar by a cell placed on the device.

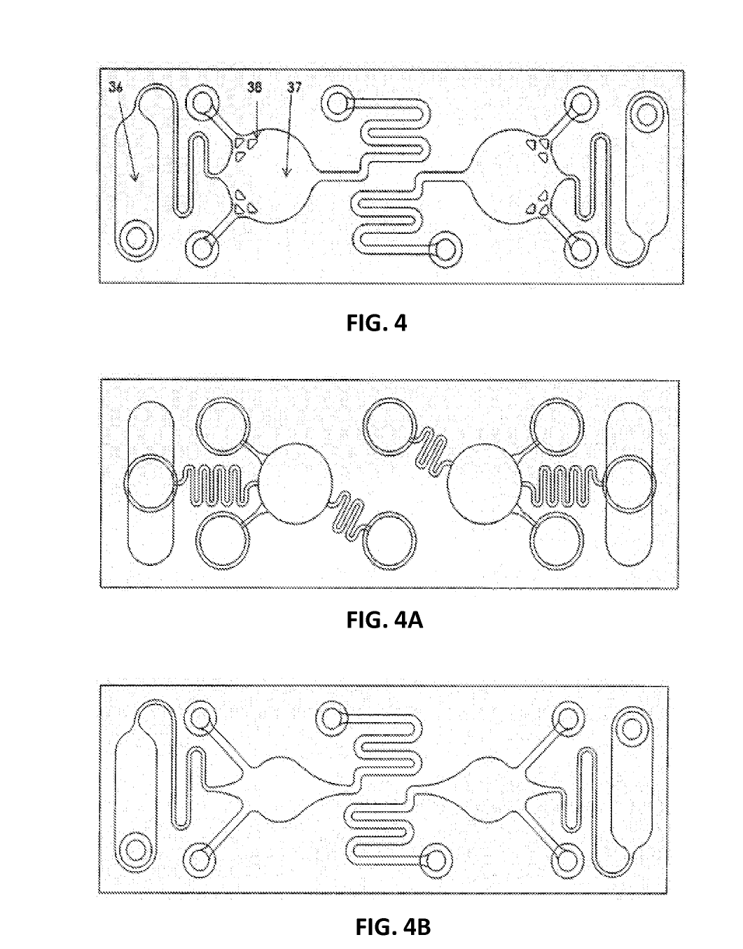

The perfusion chamber can also be adapted for hands free, long-term cell culturing purposes. With reference now to FIG. 4, the media reservoir 36 provides storage of cell culture media in an environment separate from the sample and reagent inlet, and delivers nutrients to cells placed previously in perfusion chamber 37 via passive diffusion. Baffles 38 provide a method to create even flow distribtuion into the perfusion chamber.

As will be appreciated by a person skilled in the art, the inlet placements, chamber dimensions, channel connections between the ports and perfusion chambers, and the number of perfusion chambers may take on various forms depending on the usable space available on the chip, device, or substrate. By way of examples, FIG. 3A has a hexagonal-shaped chamber as opposed to a rectangular shaped as depicted in FIG. 3; FIG. 3B has a rising channels 35 to deposit samples and reagents from the middle of the chamber; FIGS. 3A and 3B has 2 perfusion chambers as opposed to 4 perfusion chambers; FIG. 4A has a media inlet placement at the center of the media reservoir as opposed to the end of the media reservoir in FIG. 4; FIG. 4B has an elongated and smaller chamber to negate the baffles 38 and encorage even flow distribution.

### Adhesion-Based Cell Sorting

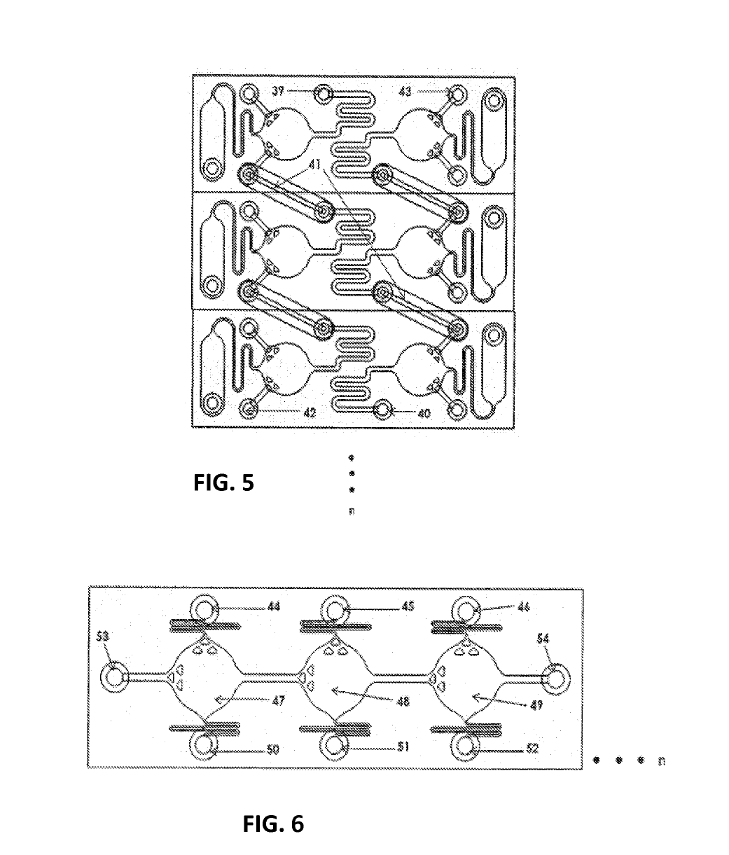

Arrays of the perfusion chamber described above can be interconnected to form a cell sorting module. With reference to FIG. 5, multiple perfusion chambers modules are linked in series. As will be appreciated by a person skilled in the art, 3 perfusion chamber modules are shown here, although more or less perfusion chambers (e.g., n) are possible. A tissue sample (which has been homogenized beforehand or can be introduced from a tissue dissociating module as described with reference to FIGS. 1, 1A and 2) consisting of a heterogeneous group of cells is introduced through cell inlet 39 and 40, and travels into the first perfusion/sorting chamber, which was initialized beforehand with a substrate with micro-and nano-features coated with proteins solutions specialized for attachment of one particular subset of cells in the heterogeneous mixture of cells in the sample. Examples of these substrates are described, for example in PCT/US2011/055444 filed Oct. 7, 2011, the contents of which are incorporated herein by reference, and examples of fabrication methods, materials, and dimensions of the micro- and nano-features are described in PCT/US2012/066162 filed Nov. 20, 2012, the contents of which are incorporated herein by reference. The cells that are not captured by the substrate in the first perfusion chamber can exit the chamber and enter the bridge connector 41, which can transfer the remaining cell population into further chambers, each containing substrates specific to a unique subset of cells in the sample. The remaining waste fluid exits through the outlet port 42 and 43. Once the desired cells are captured in each perfusion chamber, the cell sorting module can be separated by removing the bridge connectors, and each perfusion chamber can function independently for cell culturing, imaging, and metabolic assays.

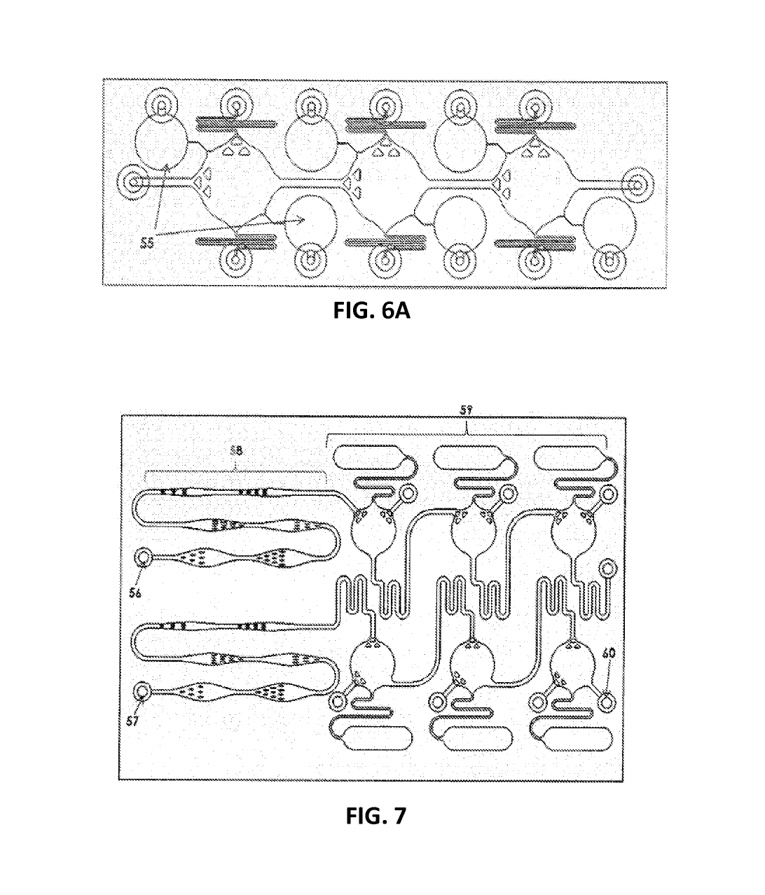

With reference now to FIG. 6, another embodiment of the cell sorting module is depicted on one microfluidic chip. Fluid containing substrates specific to different cells are introduced to the chip via inlets 44, 45, and 46, which enter the sorting chambers 47, 48 and 49 independently and coat the bottom of the chamber. Excess fluid from the substrate-coating process exit via outlet 50, 51, and 52. As will be appreciated by a person skilled in the art, 3 inlets, sorting chambers, and outlets are shown here, although more or less inlets, sorting chambers, and outlets (e.g., n) are possible. After the substrate coating step is performed, the tissue sample consisting of a heterogeneous population of cells is entered from cell inlet 53. Similar to the method described above, the first sorting chamber 46 captures a subset of cells to which the substrate is specific, and the remaining cell population flow into further chambers. The waste fluid exits via outlet 54. The attached cells can be release by introducing release agents via inlets 44, 45, and 46 and extracting from outlets 50, 51, and 52, which can be connected to an imaging stage off-chip or transferred to the perfusion chamber module as described above.

The microfluidic chip depicted in FIG. 6 can further be purposed as the perfusion module by flowing media through inlet 53. To run assays, inlets 44, 45, and 46 can be repurposed as reagent inlets, and imaging can be performed over the large sorting chambers.

### Rigid Substrate Integration

Bottom surfaces of imaging and cell sorting chambers can express different rigidities or stiffness in order to promote cell attachment and survival. These rigidities are achieved through specifically engineered biocompatible polymer gels including but not limited to polyacrylamide, polyethylene glycol, and polydimethylsiloxane. The rigidity of these polymer gels can be controlled by a number of factors such as: ratio of pre-polymer reagents, quantity of the initiator, time of polymerization, and intensity of light (e.g., intensity of light can be relevant for photo-polymerization methods). The manufacturing of these gels can be done in many mechanisms including but not limited to photo-polymerization and chemical polymerization.

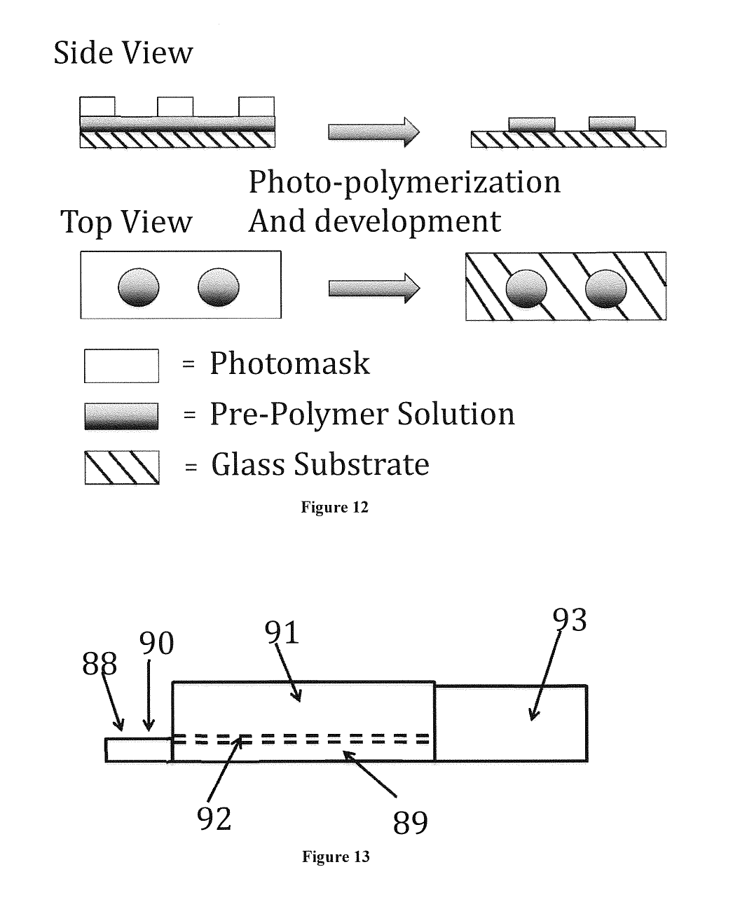

With respect to FIG. 12, an exemplary embodiment of photo-polymerization method of creating rigid substrates involves the creation of the gel substrates prior to bonding of the device is shown. In this exemplary method, the bottom layer of the microfluidic device (glass or plastic) can be silanized. Next, a solution containing the pre-polymer reagents and photoinitiator can be spin-coated onto the bottom layer. The spin-coating can achieve a uniform thickness of the solution. A photomask and light source can then be used to selectively introduce light (at the wavelength necessary for the photoinitiator) to regions of the bottom layer that correspond to the locations of the imaging or sorting chamber, which can result in polymerization of the gels only in the locations of the imaging or sorting chambers. The unpolymerized solution will be washed away during the development step leaving the bottom layer with rigid substrates in the location of the imaging chamber. The different layers of the device can then be bonded together.

In addition to the photo-polymerization method described above, a method may be used that will result in varying intensities of light being introduced to the pre-polymer solution, which can result in a gel that can have regions of different stiffness. An exemplary embodiment of this method can utilize a photomask having varying levels of transparency in the region of the imaging chamber in place of the photomask described above. The utilization of this method can allow for the gel substrate within the imaging chamber to have regions of varying stiffness for the cells to interact with.

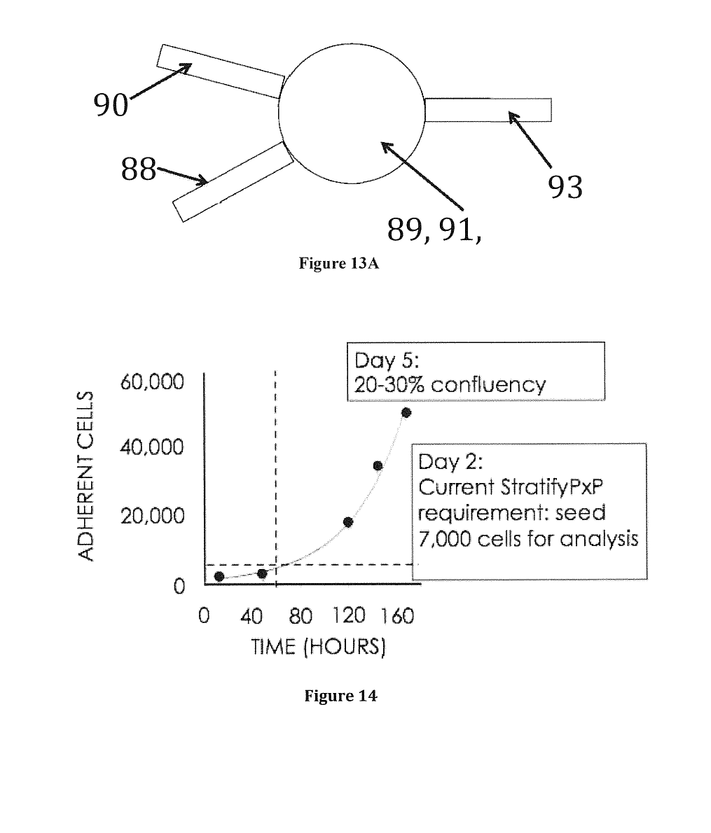

In some aspects, a polymerization method for creating rigid substrates can be achieved after a chip has already been manufactured. For example, with reference now to FIGS. 13 and 13A, which depict a top and side view of an exemplary microfluidic chip, the imaging chamber can be separated into two separate layers by a dissolvable membrane. Due to the nature of the device inlets, any injected liquids in the reagent inlet can travel to the bottom layer of the imaging chamber. To create the rigid substrate at a fixed height, a solution of pre-polymer reagents and initiators can be injected into the reagent inlet 88 until the lower imaging chamber 89 is filled. The solution can then be allowed to polymerize for a determined period of time dependent on the reagents used. Once the polymerization is completed, a solution can be injected into the upper imaging chamber 91 that will dissolve the dissolvable membrane 92 resulting in a device that is ready for use. Excess fluid can exit the chamber via waste outlet 93. An example of the solution of pre-polymer reagents and initiators is a solution containing acrylamide, bis-acrylamide, TEMED, and ammonium persulfate in order to create a poly-acrylamide gel.

### Device Integration

As noted above, though particular cell-processing functions are generally described with reference to individual cell-processing modules, it will be appreciated that the various exemplary modules and/or their functions can be integrated and/or combined to form a cell-processing system for performing multiple cell-processing functions. As will be appreciated by a person skilled in the art, all of the microfluidic device embodiments described above can be integrated in a modular fashion depending upon the desired applications of the device. By way of example, it will be appreciated that various exemplary independent modules described herein can be coupled to one another (e.g., in a lock-and-key manner) such that the microfluidic channels of each module can be coupled to one another. Alternatively, as will be discussed in detail below, various microfluidic cell-processing modules can be formed in a single monolithic structure (e.g., a microfluidic chip) to enable a specific clinical, diagnostic, and/or experimental workflow. Accordingly, the following description provides exemplary modules that can be incorporated into various systems in accord with the present disclosure. As will be appreciated by a person skilled in the art in light of the teachings herein, the various exemplary modules can be utilized and integrated in various combinations depending upon the desired applications.

For example, in one exemplary embodiment, the described modules can be fully integrated using into a microfluidic system “chip” that can be used with existing microscopy platforms. In this particular embodiment, various modules are integrated onto a 96 well plate format. As schematically depicted in FIG. 7, device operation can proceed by first introducing two samples into inlet 56 and 57. The samples then travel through zone 58 for tissue dissociation, then travel to zone 59 for adhesion-based cell sorting, cell culturing/imaging, and metabolic assays. Due to the orientation and fluidic pathway of the integrated module, the port 60 originally described as an inlet in FIG. 5 is now repurposed as a waste outlet.

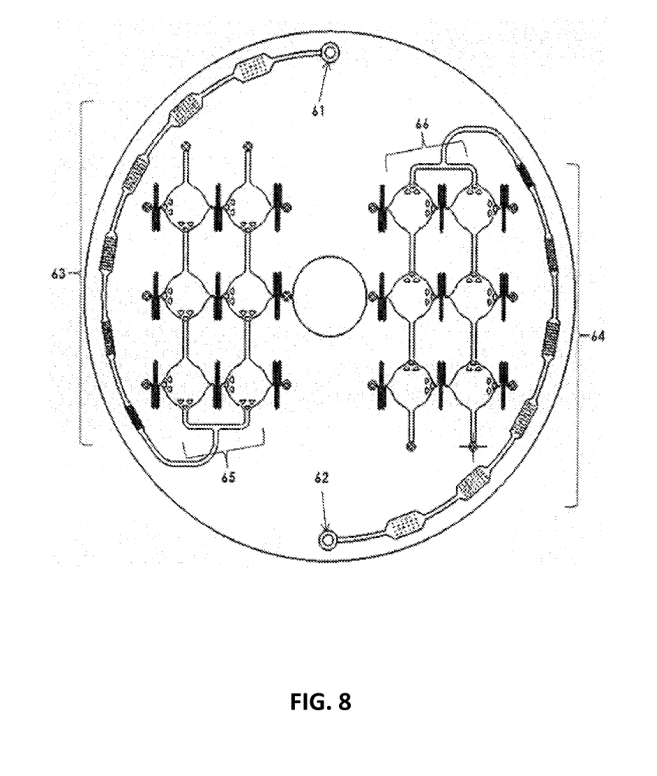

With reference now to FIG. 8, another embodiment of the integrated device is depicted on one microfluidic chip in an optical disk format. Device operation can proceed by first introducing two samples into inlet 61 and 62. The samples travels through zone 63 and 64 for tissue dissociation, then enter zone 65 and 66 for adhesion-based cell sorting, cell culturing/imaging, and metabolic assays. Two cell sorting modules are used to provide duplicate tests for one sample, and two integrated modules allow for comparison between experimental groups.

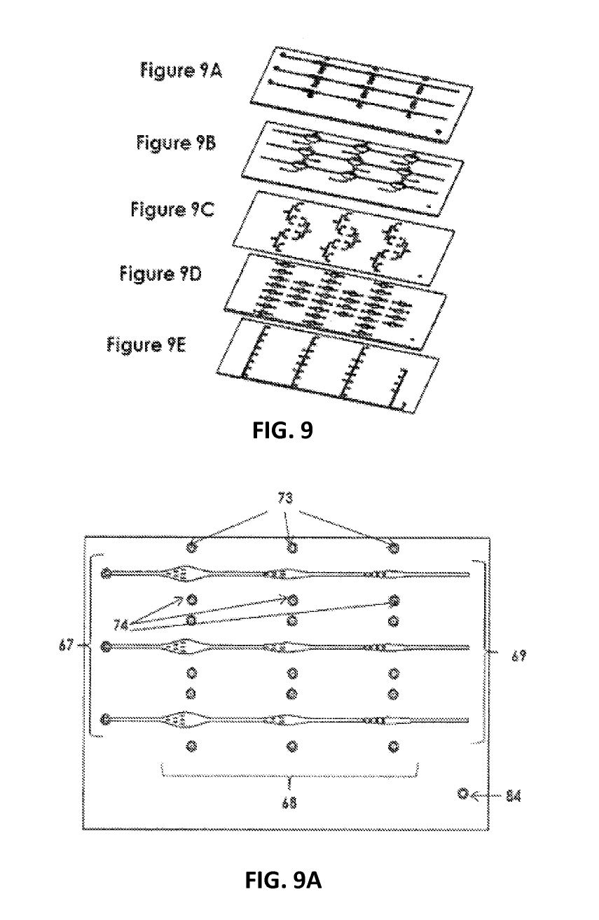

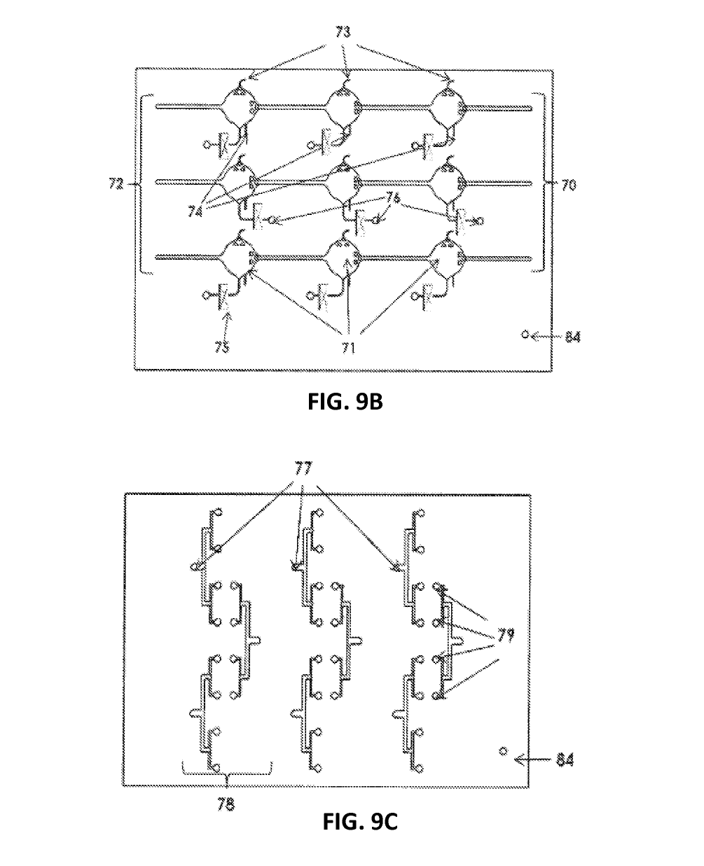

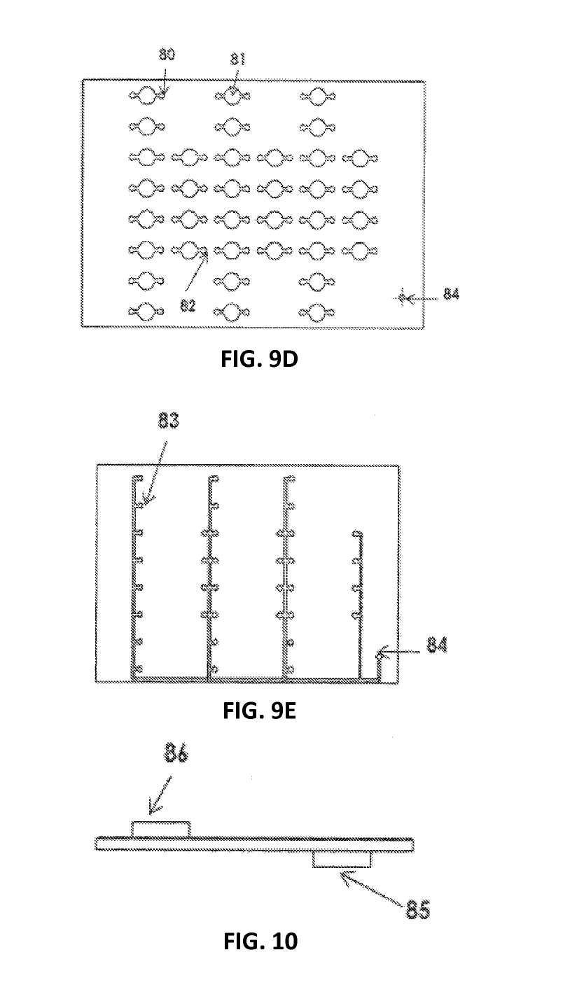

The integrated device can also exploit space in the z-direction to create a multilayer microfluidic device. In one exemplary embodiment, FIG. 9 illustrates a perspective view of the multilayer device with five different layers on a 96 well plate format. FIG. 9A-E depicts an exemplary tissue dissociation layer, cell sorting layer, flow dividing layer, cell imaging layer, and outlet layer, respectively. Device operation can proceed by first introducing samples into inlets 67. The samples enter zone 68 for tissue dissociation, then exit the tissue dissociation layer via outlets 69 and enter the cell sorting layer via cell inlets 70. As will be appreciated by a person skilled in the art, three dissociation chambers and three independent fluidic pathways are shown here, although more or less chambers and pathways can be used in accordance with the present teachings. The cells travel through sorting chambers 71 for cell sorting and brief attachment to the substrate. Excess fluid can exit the cell sorting layer via waste outlet 72. After sorting and some culturing, the cells can be detached from the substrate through introduction of cell detachment reagents such as trypsin through reagent inlets 73. After cell detachment, the detachment reagent can be removed from the sorting chamber via reagent outlets 74. Valves 75 can prevent cells from exiting the reagent outlets during the removal of detachment reagents. The cell then leaves the cell sorting layer via cell outlets 76. Three sorting chambers and associated inlets/outlets are shown in each fluidic pathway, although more or less chambers are possible. The cell outlet is connected to the flow dividing layer via cell inlet 76. The cell suspension can be distributed into flow dividing channels 78 to reduce the sample volume upon imaging. Each flow path can exit the flow channel via cell outlet 79, and into the imaging layer via cell inlet 80. Four dividing channels are shown in a diverging tree configuration, although more or less divisions in fluidic pathways and other channel configurations are possible. The cells can be fed into the cell imaging chamber 81, and can then be cultured and imaged. Each of the four replicate imaging chambers can be functionalized with the same or different substrates and micro features in order to measure different biomarkers on the same sample of cells. In one embodiment, two of the replicate imaging chambers may be functionalized with substrates of different rigidity, a third replicate imaging chamber may be functionalized with a micro-pillar array, and a fourth may be functionalized with a different ECM formulation. Excess fluid can exit the cell imaging layer and into the outlet layer via fluidic connection between waste outlets 82 in the cell imaging layer and waste inlets 83 in the outlet layer. The waste fluid can be collected in a common channel and removed from the chip via outlet 84.

The various microfluidic layers can be fluidically coupled via reversible or irreversible connections between inlets and outlets. By way of example, press fit ports 85 and 86 are depicted in FIG. 10, allowing for addition and removal of a fluidic layer during operation of the device.

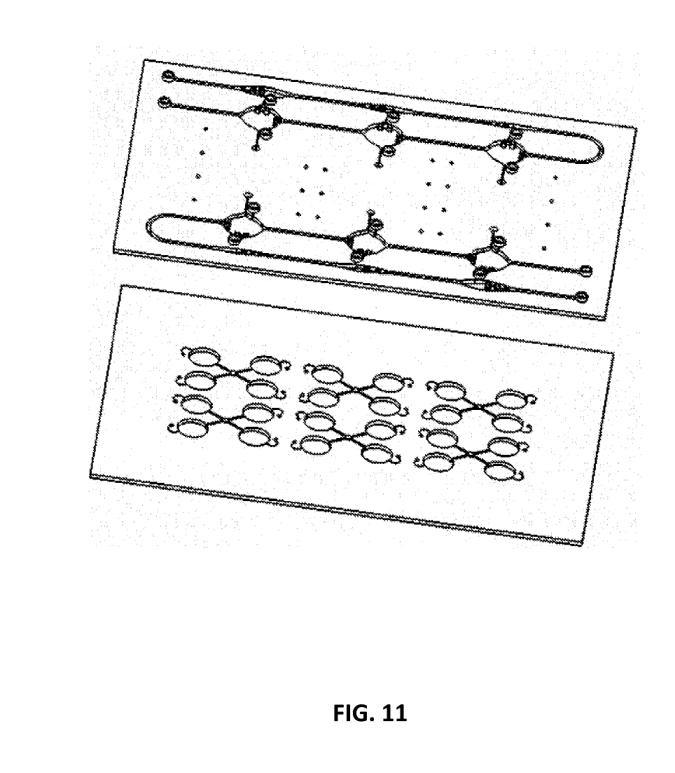

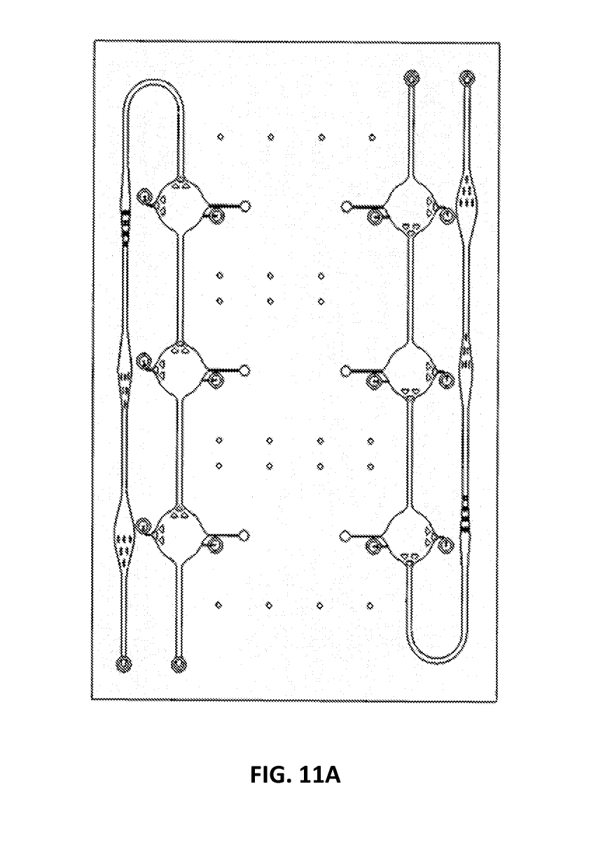

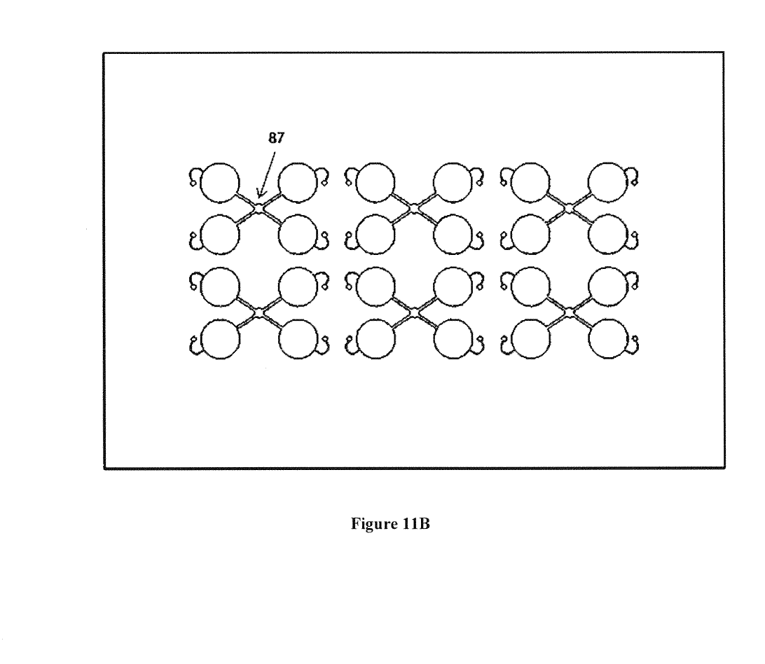

As will be appreciated by a person skilled in the art, the functional features found on the multilayer device can take on various configurations and number of features depending on operational need and available space on the chip. By way of example, FIG. 11 depicts an integrated multilayer microfluidic device with two functional layers as opposed to five layers as depicted in FIG. 9, the top layer being a combination of tissue dissociation and cell sorting functionalities, and the bottom layer being a combination of the flow dividing layer, cell imaging layer, and outlet layer. With reference to FIG. 11A, two independent fluidic pathways are shown as opposed to three in FIG. 9A. With reference to FIG. 11B, the flow divider 87 takes on a radial configuration as opposed to the diverging tree configuration as depicted in the flow dividing channels 77 in FIG. 9C.

In various embodiments discussed above, given the inputs of mammalian tissue, the device, in an automated, systematic fashion, can dissociate, segregate, sort, enrich, manipulate, and assay cells for biomarker quantification. These quantified biomarkers, which can be based on physical properties of the cells or biochemical/metabolic properties of the cells or associated extracellular components, can then be used as inputs into algorithms to output quantifiable metrics regarding the aggressiveness, or oncogenic potential, of a cancer, or the invasion, motility, or metastatic potential of a cancer. Examples of these algorithms can be found, for example in PCT/US2011/055444 filed Oct. 7, 2011, the contents of which are incorporated herein by reference.

The present inventors have developed innovative microfluidic devices. Based on the quantification of biomarkers in such devices, metrics of Oncogenic Potential and Metastatic Potential were developed to aid physicians in treatment decisions and supplement the qualitative Gleason score with a sensitive, specific, and quantitative metrics. The devices and methods described and contemplated herein represent, for example, a personalized diagnostic solution capable of predicting aggressiveness to better guide therapy selection. Moreover, the inventors have cultured prostate cells from clinically relevant patient samples in vitro.

The presently described devices, methods and clinical measures can, in certain embodiments, be utilized along with the traditional Gleason Scores in evaluating patients, which adds critical information to the evaluation of patients having Gleason scores of, for example, 6-8. OP and MP allow the presently described technology to mitigate the current state of over-treatment in prostate cancer, inform the choice between local and systemic therapy, and identify aggressive tumors earlier during watchful waiting or active surveillance periods.

On one exemplary protocol, biopsied cells are introduced (e.g., injected) into microfluidic devices of the present disclosure. The cells are then analyzed on the chip using, for example, automated light/fluorescent microscopy, and images are uploaded to, or accessed in a database by, a program that utilizes machine vision image analysis to calculate and return OP and MP values. In such an exemplary protocol, the following steps are characterized the use of one or more technologies selected from the group consisting of ECM formulation, a microfluidic device, a biomarker suite, machine vision software, and prognostic algorithms. Frequently, raw images are generated that require processing. After processing and then analysis, the resulting data is often synthesized into distinct, meaningful outputs that can be delivered to physicians. Though prostate samples are often utilized, the presently described technologies and methods are readily applied to bladder, kidney, breast, colon, and lung tissues and cells.

In one exemplary protocol, a patient sample is processed as noted above and OP and MP values or information are provided to a physician within about five days in addition to confidence intervals to gauge the sensitivity for each patient's results (e.g., from a biopsy). Thereafter, the physician can provide more informed treatment options to patients with increased confidence (e.g., radical prostatectomy or active surveillance).

In certain embodiments, the present devices and methods provide the ability to differentiate between low-risk (low-grade) and high-risk (high-grade) prostate cancer as correlated with the reference standard of the Gleason Score. The present devices and methods also often provide a stratification of low-risk, intermediate-risk, and high-risk patients as correlated with the reference to Gleason Score standards. In addition, the present devices and methods provide the ability to differentiate between different types of intermediate risk patients (Gleason 6 or 7)—risk stratifying within the intermediate patient prostate cancer population, segregating patients as having indolent, locally aggressive, or metastatically aggressive types of cancer. Also, the present devices and methods provide the ability to act as a therapy guide, differentiating patients who should be treated via active surveillance, surgery or radiation, and/or adjuvant therapy. In certain embodiments, the present devices and methods also provide the ability to facilitate compound validation and therapeutic pipeline acceleration to bridge conversations with strategics towards exploratory and co-development deals. In frequent embodiments, the present devices and methods also provide the ability to distinguish between normal and cancer samples, predict aggressive potential of disease, stratify patients by risk category, wthin patients that are intermediate risk (clinically ambiguous), identify patients with local growth potential and/or metastatic potential, control for biopsy sample heterogeneity, provide high signal to noise biomarker analysis, and return clinically actionable metrics

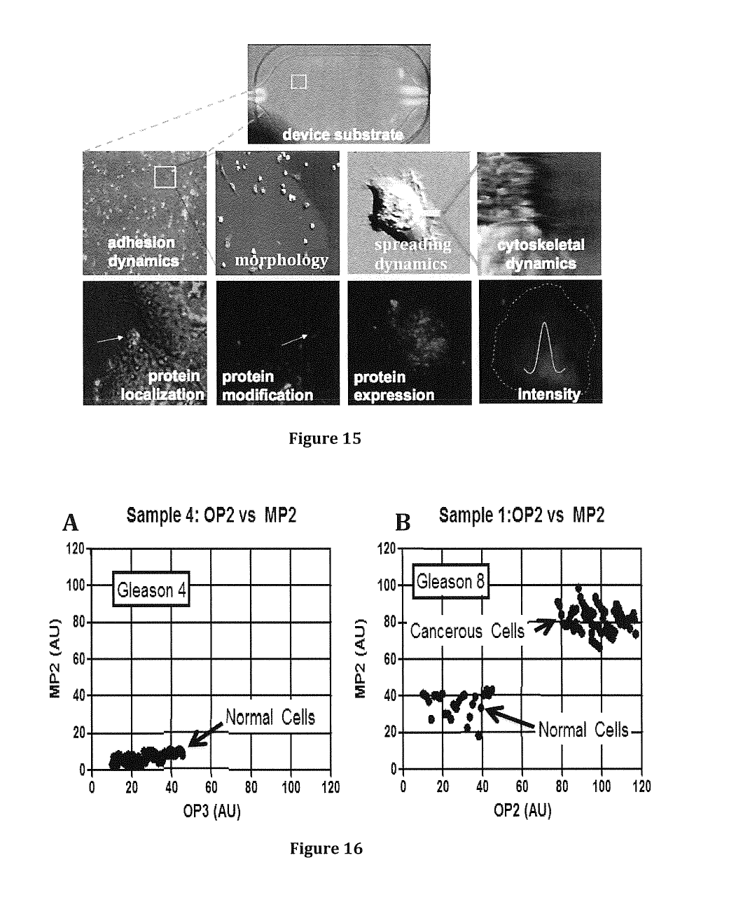

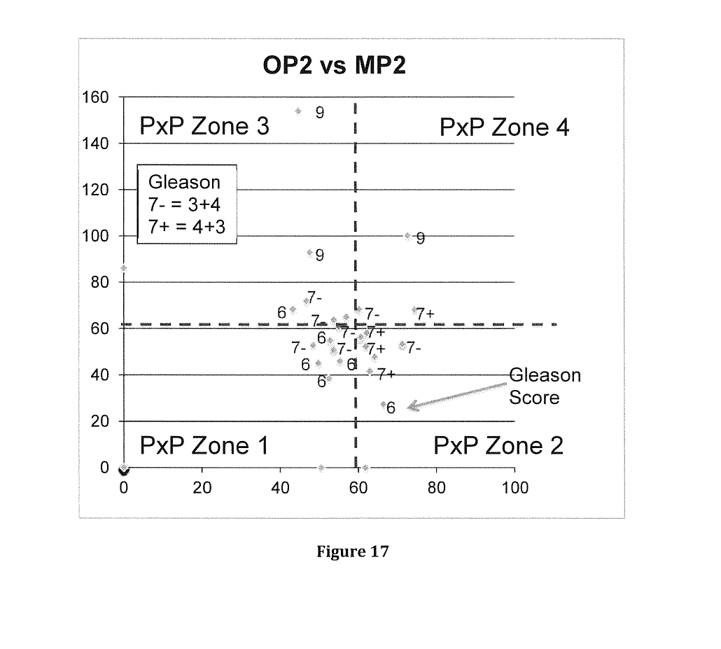

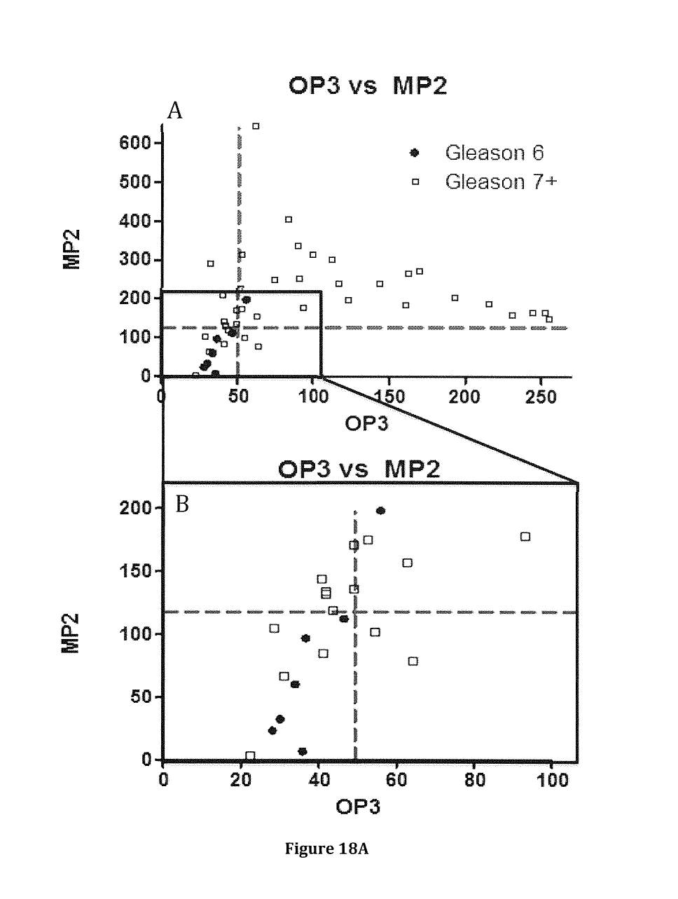

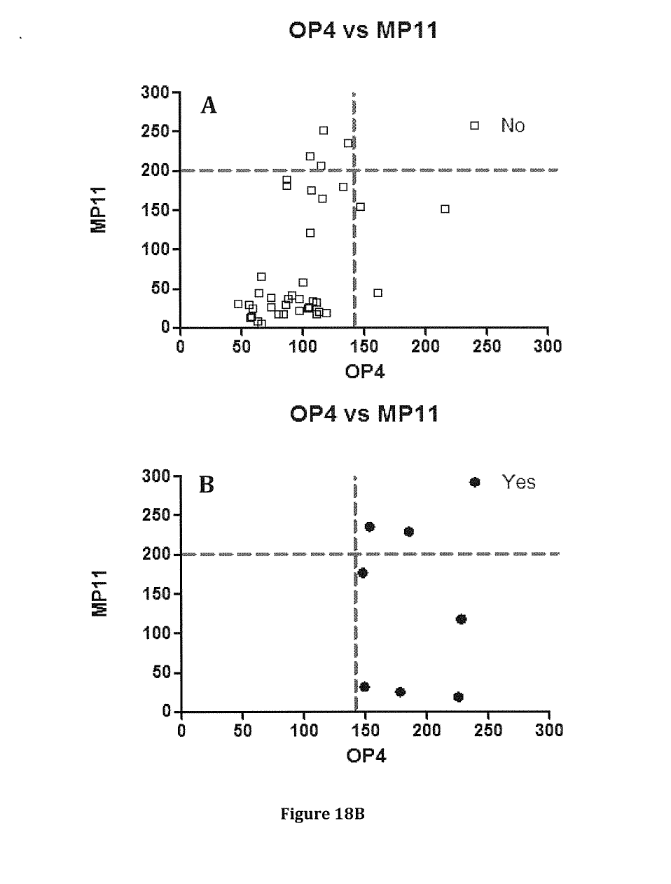

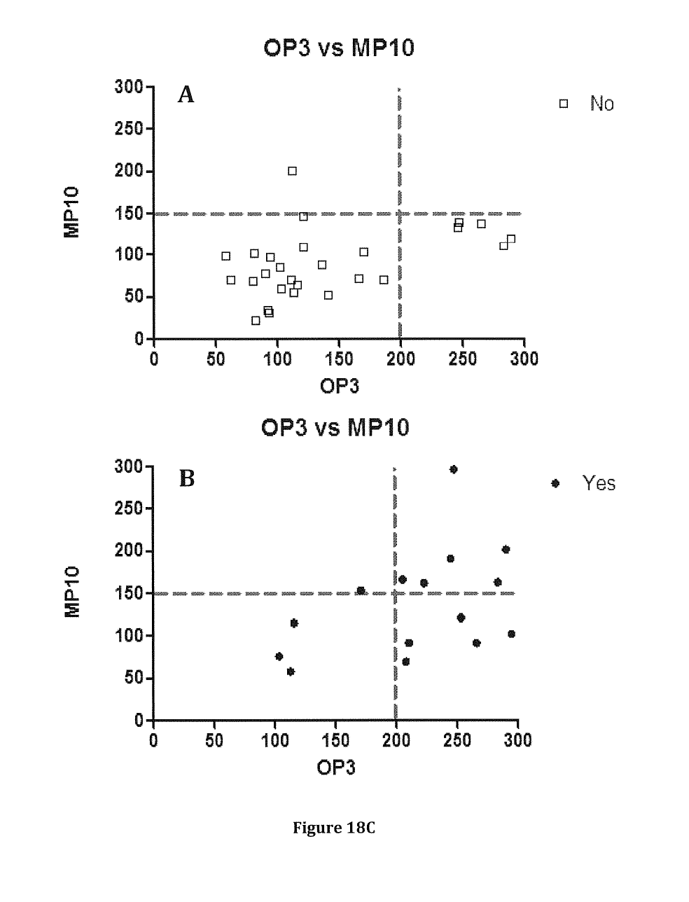

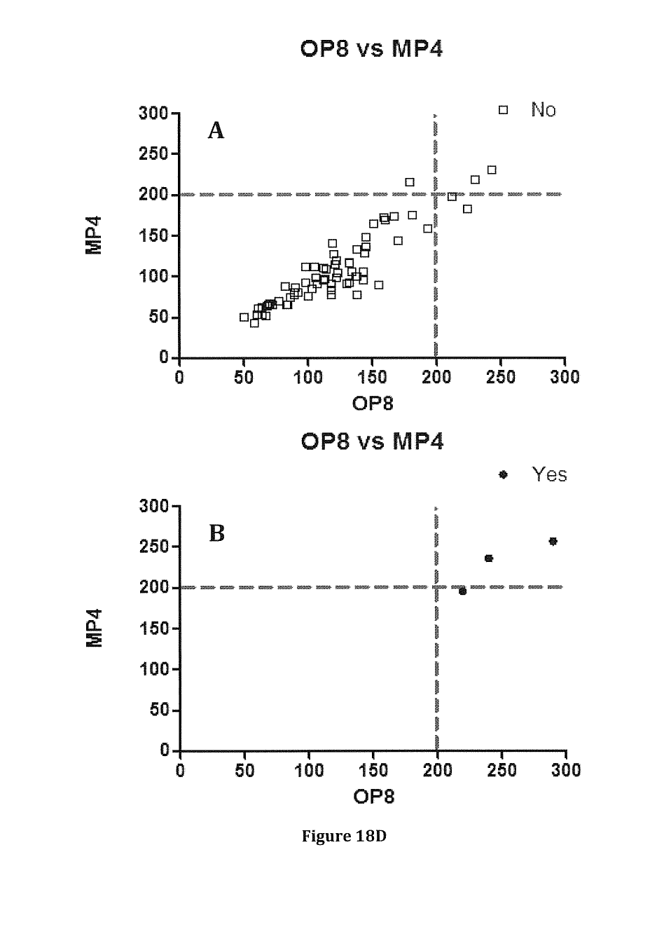

The microfluidic chips and related methods of the present disclosure have been successfully applied to diagnostic processes in the clinic. By way of example, FIGS. 14-18D provide results at various stages of the diagnostic process obtained from samples run through an exemplary microfluidic device. FIG. 14 demonstrates the microfluidic device's ability to prepare the sample for analysis before any in vitro transformation occurs. FIG. 15 provide select images of various biomarkers obtained from the diagnostic process operated within an exemplary device. With reference to FIG. 16, the oncogenic potential (OP) and metastatic potential (MP) metrics derived from biomarkers obtained from within an exemplary device can distinguish the difference in cancerous/non-cancerous cells distribution between normal and malignant tissue. FIG. 17 to FIG. 18B depict the ability of representative sample diagnostic results according to the present disclosure to be translated to relevant patient clinical information with various iterations of OP and MP. FIG. 17, for example, demonstrates the ability of devices and methods according to the present disclosure to stratify patients into 4 zones that predict indolent (PxP Zone 1), local growth potential (PxP Zone 2), metastatic potential (PxP Zone 3), and both local growth and metastatic growth potential (PxP Zone 4). FIG. 18A is an example of the OP3-prime and MP2-prime algorithms distinguishing Gleason 6 samples from Gleason 7s, 8s, and 9s with 79.5% sensitivity and 85% specificity (n=56). FIG. 18B demonstrate the OP4 and MP11 algorithms's ability to predict samples that will invade seminal vesicles. FIG. 18C is an example of the OP3 and MP10 algorithms predicting samples that will exhibit positive margins during surgery. Finally, FIG. 18D demonstrates the ability of the OP8 and MP4 algorithms to predict samples that will exhibit vascular invasion, resulting in metastasis into the bloodstream. Further examples of OP and MP metrics can be found, for example in PCT/US2011/055444, filed Oct. 7, 2011, the contents of which are incorporated herein by reference. In frequent embodiments, a microfluidic device is provided for processing tissue, comprising: a cell inlet port for receiving a tissue fragment, a cell dissociation chamber comprising a plurality of microstructures, an outlet port for extracting a cell suspension, a channel fluidly coupled to the inlet port, the chamber, and the outlet port to allow sequential flow through the device, and a pump coupled to the inlet port and/or outlet port to cause displacement of a fluid through the channel and chamber.

In other frequent embodiments, a microfluidic device is provided for processing tissue, comprising: a cell inlet port for receiving a tissue fragment, a cell dissociation chamber comprising a plurality of microstructures, a plurality of pressure inlet ports for circulating a fluid back and forth within the cell dissociation chamber; an outlet port for extracting a cell suspension, a channel fluidly coupled to the inlet port, the chamber, and the outlet port to allow controlled flow of the fluid through the device, and a pump coupled to the cell inlet port, pressure inlet ports, chamber, and/or outlet ports to circulate the fluid back and forth through the dissociation chamber.

In certain embodiments the microstructures comprise posts and/or are diamond or rectangular in shape. Often the device comprises two or more cell dissociation chambers, wherein each of the cell dissociation chambers comprises a plurality of microstructures having a differing gap distances. Often, each of the plurality of the microstructures is separated from the other microstructures by a distance defined as a gap distance, and wherein the chamber comprises multiple gap widths. Frequently, the gap distance is between 1 micron and 1 millimeter in distance.

In certain embodiments a microfluidic device is provided for processing tissue, comprising: a cell inlet port for receiving a tissue fragment and/or a cell suspension, a perfusion chamber for culturing, imaging, and/or assaying a cell, a reagent inlet for receiving assay reagents, the reagent inlet being in fluid communication with the perfusion chamber, an outlet for extracting excess fluid, a channel fluidly coupled to the cell inlet, perfusion chamber, reagent inlet, and outlet port to allow controlled flow through the device, and a pump coupled to the cell inlet and/or the reagent inlet to cause displacement of fluid through the channel, chamber, and the outlet. Often the perfusion chamber comprises a cell adhesion surface. Also often, the device further comprises a perfusion layer comprising a channel disposed therein and positioned relative to the cell adhesion surface to allow diffusion of a gas and/ora nutrient to a cell adhered to the cell adhesion surface. The cell adhesion surface is optionally functionalized with a reagent suitable to facilitate a preferential adhesion of a cell to the surface. Often the reagent comprises one or more of fibronectin, collagen, laminin, or vitronectin. The device itself may be comprised of a thermoplastic, a thermoset, or an elastomer. Often a device composition material comprises an epoxy, a phenolic, polydimethylsiloxane (PDMS), glass, silicone, nylon, polyethylene, and/or polysterene.

In frequent embodiments, the perfusion chamber comprises an optically transparent portion, wherein the optically transmissive portion is positioned relative to the cell adhesion surface to permit optical interrogation of a cell adhered to the cell adhesion surface. Frequently, a plurality of perfusion chambers are provided that are fluidly coupled. Often a surface of the optically transparent portion is functionalized with a reagent suitable to prevent adhesion of a cell to the surface. The cell adhesion surface is optionally planar or substantially planar. Frequently, the cell adhesion surface comprises a microstructure.

In certain embodiments the device further comprises a media reservoir in fluid communication with the perfusion chamber to passively diffuse a nutrient and/or reagent (or multiple nutrients or reagents) into the perfusion chamber.

In certain embodiments a plurality of perfusion chambers are fluidly coupled by bridge connectors. Often, the bridge connectors are removable from the device.

In certain embodiments, each of the plurality of perfusion chambers comprises a cell adhesion substrate, wherein each substrate is configured to selectively capture a designated subset of cells within a heterogeneous cell population in the sample. Often, the cell adhesion substrate comprises a microstructure and/or a protein formulation configured to preferentially capture a designated subset of cells.

In certain embodiments, a microfluidic device is provided for processing tissue and/or cells, comprising: a reagent inlet for introducing a reagent input, a sorting chamber for selectively capturing a designated subset of a heterogeneous cell population culturing, imaging, and/or assaying one or more cells, a reagent outlet for extracting excess fluid introduced into the reagent inlet, a cell inlet for introducing a cell suspension, a cell outlet for extracting excess fluid from the cell inlet, a channel in fluid communication with thethe reagent inlet, cell inlet, sorting chamber, reagent outlet, and cell outlet for controlling and/or confining fluid flow therethrough, and a pump coupled to the reagent inlet and cell inlet to cause displacement of a fluid through the channel, chamber, reagent outlet, and cell outlet. Often, a substrate, a release reagent, and/or an assay reagent are introduced to or comprised in the reagent input.

In certain embodiments, the sorting chamber is configured to culture, image, and/or assay one or more cells.

In one embodiment, a microfluidic device is provided for processing tissue and/or cells, comprising: a tissue dissociation module, a cell sorting module, a channel fluidly coupled to the tissue dissociation module and the cell sorting module for allowing sequential flow therethrough, and a pump for effecting displacement of a fluid through the tissue dissociation module and cell sorting module. Often, the tissue dissociation module comprises: a cell inlet port for receiving a tissue fragment, a cell dissociation chamber comprising a plurality of microstructures, and an outlet port for extracting a cell suspension, wherein the channel is fluidly coupled to the outlet port. Also often, the pump is configured to provide an unidirectional flow of the fluid through the cell dissociation chamber. In certain embodiments, the pump is configured to circulate the fluid back and forth through the cell dissociation chamber.

In certain embodiments, the cell sorting module comprises: a cell inlet port for receiving a cell suspension from the tissue dissociation module, a perfusion chamber for culturing, imaging, and/or assaying a cell, a reagent inlet for receiving an assay reagent, the reagent inlet being in fluid communication with the perfusion chamber, an outlet for extracting excess fluid from the perfusion chamber.

In certain embodiments, the perfusion chamber comprises a cell adhesion surface. Often the cell adhesion surface is functionalized with a reagent suitable to facilitate preferential adhesion of the cell to the surface.

In certain embodiments, the tissue dissociation module comprises a plurality of perfusion chambers fluidly coupled to one another. Often, each of the plurality of perfusion chambers comprises a cell adhesion substrate, wherein each substrate is configured to selectively capture a designated subset of cells within a heterogeneous cell population in the sample.

In certain embodiments, a microfluidic device is provided for processing tissue and/or cells, comprising: an inlet for receiving an input, one or more, or two or more, layers selected from the group consisting of: a tissue dissociation layer, a cell sorting layer, a flow dividing layer, an imaging layer, and an outlet layer, a plurality of microfluidic channels connecting the two or more layers for allowing fluid flow between the layers, and an outlet for extracting an output. Frequently, the layers are vertically arranged or positioned relative to one-another. Often in such arrangements, one layer partially or completely overlaps another layer of the same device. The microfluidic channels often act as fluid conduits connecting multiple layers on the same or different vertical levels. Frequently, the plurality of microfluidic channels connecting the two or more layers allows for reversible fluid communication therebetween. Often, the plurality of microfluidic channels connecting the two or more layers allows for one-way fluid flow therebetween. Also often, the device further comprises a pump for causing displacement of a fluid through the two or more layers. The pump can optionally be coupled to a sample inlet of the tissue dissociation layer to cause displacement of a fluid through the two or more layers.

Often, the device comprises multiple vertically arranged layers, including the tissue dissociation layer, the cell sorting layer, the flow dividing layer, the imaging layer, and the outlet layer. Also often, the device comprises two or more of the tissue dissociation layer, the cell sorting layer, the flow dividing layer, the imaging layer, or the outlet layer. In frequent embodiments, the device comprises a plurality of vertically arranged layers, each vertically arranged layer comprising two or more of the tissue dissociation layer, the cell sorting layer, the flow dividing layer, the imaging layer, and/or the outlet layer.

Often, the tissue dissociation layer comprises: a cell inlet port for receiving a tissue fragment, a cell dissociation chamber comprising a plurality of microstructures, and an outlet port for extracting a cell suspension, wherein at least one of the microfluidic channels is fluidly coupled to the outlet port. Often ,the cell sorting layer comprises: a cell inlet port for receiving a cell suspension from, if present, the tissue dissociation layer, another layer, or the inlet, a perfusion chamber for sorting a cell, a reagent inlet for receiving an assay reagent, the inlet being in fluid communication with the perfusion chamber, an outlet for extracting excess fluid from the perfusion chamber. Also often, the cell inlet port of the cell sorting layer is fluidly coupled to an outlet port of the tissue dissociation layer.

In certain embodiments, the device further comprises a valve configured to control the flow of a fluid through the cell sorting layer and optionally a cell outlet for extracting sorted cells.

Often, the flow dividing layer comprises: a cell inlet port for receiving a suspension of sorted cells from, if present, the cell sorting layer, another layer, or the inlet, a flow divider for reducing a sample volume, a cell outlet for extracting cells, and a channel for fluidic coupling the cell inlet, flow divider, and cell outlet for controlling fluid flow therethrough.

Also often, the imaging layer comprises: a cell inlet port for receiving a suspension of sorted cells, an imaging chamber for imaging cells disposed therein, a waste outlet for extracting a waste fluid, and a channel for fluidic coupling the cell inlet, imaging chamber, and waste outlet for controlling fluid flow therethrough. In certain embodiments, the device further comprises a reagent inlet for introducing a reagent to a cell within the imaging chamber.

Often, the outlet layer comprises: a waste inlet for receiving a waste fluid generated in the tissue dissociation layer, the cell sorting layer, the flow dividing layer, and/or the imaging layer, a waste outlet for removing the waste fluid from the microfluidic device, and a channel in fluid communication with the waste inlet and the waste outlet for controlling or containing fluid flow therethrough. Often the device further comprises a waste reservoir for storing the waste fluid disposed between the waste inlet and the waste outlet.

In certain embodiments, a method of manufacturing a device described herein is provided, comprising: producing a rigid substrate within the device or portion thereof having a fixed height. Often, the rigid substrate comprises a plurality of microstructures. Frequently, the rigid substrate is produced within the device or portion thereof, including surfaces of modules, channels, or layers thereof, using photo-polymerization. Often, the rigid substrate is produced having regions of different stiffness within the device or portion thereof by modulating an intensity of light during a photo-polymerization process. In certain embodiments, the device comprises an imaging chamber or layer, and the method comprised producing a rigid substrate in the imaging chamber or layer through the use of a dissolvable membrane. The imaging chamber or layer often comprises a two-layer imaging chamber or layer. In certain embodiments, such devices are configured in an optical disc format.

In frequent embodiments, methods are provided for evaluating a cell, tissue, or patient, comprising introducing a cell to a microfluidic device functionalized with an extracellular matrix formulation, imaging the cell, and stratifying the cell based on oncologic potential and metastatic potential. Machine vision is often utilized to image the cell. Also, often the cell is exposed to a biomarker or suite of biomarkers in the device. In certain embodiments the evaluation comprises determining the potential of the cell to invade a seminal vesicle, determining the potential of the cell to invade the vasculature of a patient, or determining the likelihood that a tumor from which the cell was derived will exhibit positive margins during surgery. In certain embodiments, a method is provided for evaluating a cell, tissue, or patient, comprising introducing a cell to a microfluidic device described hereinabove functionalized with an extracellular matrix formulation, imaging the cell, stratifying the cell based on oncologic potential and metastatic potential, and/or stratifying the cell based on or in reference to a Gleason score. In the methods of the present invention, assaying cell types such as prostate, colon, lung, bladder, kidney, and/or breast cells or cellular extracts or components thereof is contemplated.

One skilled in the art will appreciate further features and advantages of the presently disclosed methods, systems and devices based on the above-described embodiments. Accordingly, the presently disclosed methods, systems and devices are not to be limited by what has been particularly shown and described, except as indicated by the appended claims. All publications and references cited herein are expressly incorporated herein by reference in their entirety.

1-71. (canceled)

72. A microfluidic device for processing tissue, comprising:
a cell inlet port for receiving a tissue fragment and/or a cell suspension,
a perfusion chamber comprising a cell adhesion surface for culturing, imaging, and/or assaying a cell, wherein the perfusion chamber comprises an optically transparent portion, wherein the optically transmissive portion is positioned relative to the cell adhesion surface to permit optical interrogation of a cell adhered to the cell adhesion surface
a reagent inlet for receiving assay reagents, the reagent inlet being in fluid communication with the perfusion chamber,
an outlet for extracting fluid,
a channel fluidly coupled to the cell inlet, perfusion chamber, reagent inlet, and outlet port to allow controlled flow through the device, and
a pump coupled to the cell inlet and/or the reagent inlet to cause displacement of fluid through the channel, chamber, and the outlet.

73. The microfluidic device of claim 72, further comprising a perfusion layer comprising a channel disposed therein and positioned relative to the cell adhesion surface to allow diffusion of a gas and/or a nutrient to a cell adhered to the cell adhesion surface.

74. The microfluidic device of claim 72, wherein a surface of the optically transparent portion is functionalized with a reagent suitable to prevent adhesion of a cell to the surface.

75. The microfluidic device of claim 72, wherein the cell adhesion surface comprises a microstructure and/or a protein formulation configured to preferentially capture the designated subset of cells.

76. The microfluidic device of claim 72, further comprising a plurality of perfusion chambers.

77. The microfluidic device of claim 76, wherein each of the plurality of perfusion chambers comprises a cell adhesion substrate, wherein each substrate is configured to selectively capture a designated subset of cells within a heterogeneous cell population in the sample.

78. The microfluidic device of claim 72, further comprising a cell dissociation chamber comprising a plurality of microstructures, and wherein the microstructures comprise diamond or rectangular shaped posts.

79. The microfluidic device of claim 78 comprising two or more cell dissociation chambers, wherein each of the cell dissociation chambers comprises a plurality of microstructures having a differing gap distances.

80. The microfluidic device of claim 79, wherein the gap distance is between 1 micron and 1 millimeter in distance.

81. The microfluidic device of claim 72, wherein the perfusion chamber comprises a sorting chamber for selectively capturing a designated subset of a heterogeneous cell population culturing, imaging, and/or assaying one or more cells.

82. The microfluidic device of claim 81, wherein the sorting chamber is configured to culture, image, and/or assay one or more cells.

83. The microfluidic device of claim 78, wherein the perfusion chamber is comprised in an imaging layer, the cell dissociation chamber is comprised in a tissue dissociation layer, and the imaging layer and tissue dissociation layers are distinct layers of the device.

84. The microfluidic device of claim 83, further comprising:
a cell sorting layer,
a flow dividing layer,
an outlet layer,
a plurality of microfluidic channels connecting the two or more layers for allowing fluid flow between the layers, or
an outlet for extracting an output.

85. The microfluidic device of claim 83, wherein the tissue dissociation layer comprises:
a cell inlet port for receiving a tissue fragment,
a cell dissociation chamber comprising a plurality of microstructures, and
an outlet port for extracting a cell suspension,
wherein at least one of the microfluidic channels is fluidly coupled to the outlet port.

86. The microfluidic device of claim 84, wherein the cell sorting layer comprises:
a cell inlet port for receiving a cell suspension from, if present, the tissue dissociation layer, another layer, or the inlet,
a perfusion chamber for sorting a cell,
a reagent inlet for receiving an assay reagent, the inlet being in fluid communication with the perfusion chamber,
an outlet for extracting excess fluid from the perfusion chamber.

87. The microfluidic device of claim 84, wherein the flow dividing layer comprises:
a cell inlet port for receiving a suspension of sorted cells from, if present, the cell sorting layer, another layer, or the inlet,
a flow divider for reducing a sample volume,
a cell outlet for extracting cells, and
a channel for fluidic coupling the cell inlet, flow divider, and cell outlet for controlling fluid flow therethrough.

88. The microfluidic device of claim 83, wherein the imaging layer comprises:
a cell inlet port for receiving a suspension of sorted cells,
an imaging chamber for imaging cells disposed therein,
a waste outlet for extracting a waste fluid, and
a channel for fluidic coupling the cell inlet, imaging chamber, and waste outlet for controlling fluid flow therethrough.

89. The microfluidic device of claim 84, wherein the outlet layer comprises:
a waste inlet for receiving a waste fluid generated in the tissue dissociation layer, the cell sorting layer, the flow dividing layer, and/or the imaging layer,
a waste outlet for removing the waste fluid from the microfluidic device, and
a channel in fluid communication with the waste inlet and the waste outlet for controlling or containing fluid flow therethrough.

90. The method of claim 83, wherein the imaging chamber or layer comprises a multiple layer imaging chamber or layer.

91. A method of manufacturing the device of claim 72, comprising producing a rigid substrate within the device or portion thereof having a fixed height.

92. A method of evaluating a cell, comprising introducing a cell to the microfluidic device of claim 72 functionalized with an extracellular matrix formulation, imaging the cell, and stratifying the cell based on oncologic potential and metastatic potential or other biologically or clinically relevant output.

93. A method of conducting a live cell analysis on a microfluidic device, comprising introducing a tissue sample to the device of claim 78, and on the device: dissociating and sorting individual cells from the tissue sample, culturing the individual cells, and imaging the individual cells.
